(19) United States
(12) Patent Application Publication
(10) Pub. No.: US 2024/0404643 A1
(43) Pub. Date: Dec. 5, 2024

(54) System and method for deep genomic compression

(71) Applicant: Divon Mordechai Lan, Bangkok (TH)

(72) Inventor: Divon Mordechai Lan, Bangkok (TH)

(21) Appl. No.: 18/204,466

(22) Filed: Jun. 1, 2023

(51) Int. Cl. G16B 50/50; H03M 7/30

(52) U.S. Cl. CPC G16B 50/50; H03M 7/6005; H03M 7/6011

(57) ABSTRACT

The present invention relates to a novel method employing an enhanced approach towards compression of a collection of unaligned and aligned genomic data, exploiting information redundancies between unaligned and aligned genomic data. This approach is based on four modules, wherein the method provides various technological advancements that have not been employed before.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention is in the field of bioinformatics. Specifically, it deals with the compression of unaligned and aligned genomic data. This unaligned data is often stored in files in FASTQ format (de facto standard) and aligned data is often stored in SAM or BAM format, and the files are typically very large (GBs to hundreds of GB per file). Thus, compressing these files is often beneficial.

### Description of the Related Art

A genome is the totality of genetic material carried by an organism. A genome consists of DNA or RNA and includes both genes in the coding regions as well as noncoding regions. A genome can be described by a sequence which is a complete list of the nucleotides (e.g., A, C, G, and T for DNA genomes, and other characters, such as N, describing ambiguity) that make up all the chromosomes of an individual or a species. Genomic data includes genomic sequences that originate from one or more genomes, as well as from other natural or artificial genetic material such as RNA, and are possibly modified naturally (e.g. by degradation) or artificially for the purpose of experimentation (e.g. treated with bisulfite).

As the number of genomes being identified grows, the ability to retain the information is being strained. Currently, the number of genomes is rising rapidly. However, the cost of data storage space to store data corresponding to identified genomes is not falling at a similar rate. If data compression and transmission efficiency can be improved, millions of dollars in storage and transmission costs can be saved.

A common approach for effectively compressing genomic files is referred to hereinafter as context-based compression. The context-based compression technique involves the utilization of a computer system equipped with specialized software. The software operates by parsing the source genomic file, such as FASTQ or SAM/BAM/CRAM, and partitioning the data encompassed within the genomic data into distinct contexts. Subsequently, an appropriate compression method is selectively employed for each specific context.

By way of illustration, a FASTQ file can be partitioned into discrete contexts, including the read name, sequence data, and base quality score data. To restore the original file, a corresponding decompression software is employed, which performs the inverse operation. Firstly, the decompression software decompresses each context individually and subsequently combines them in a coherent manner to reconstruct the original file.

By looking at prior art multiple advancements have been seen in similar regards. For instance, a CN patent 1,106,74094B relates to method, system and medium for no-reference sequence compression and decompression of SAM and BAM files. The invention discloses a method, a system and a medium for compressing, decompressing and restoring a non-reference sequence of a SAM or BAM file, wherein the method for compressing the non-reference sequence comprises the steps of traversing and reading the SAM or BAM file to obtain a current line, performing domain cutting, and restoring a real reference sequence corresponding to the current line according to CIGAR domain data of the current line and SEQ domain data; updating the restored real reference sequence to the corresponding position of the current row in the shadow reference sequence, grouping the current row to calculate difference information and compressing the difference information to a target compressed file, and finally compressing the shadow reference sequence to the target compressed file; the decompression method is the inverse operation of the compression method.

A WO patent 2,022,125754A1 relates to computational method and system for compression of genetic information. To reduce the total amount of linear sequence (DNA, RNA or other medium) required to encode a set of genetic elements, this patent describes a computational method for compressing genetic information by finding one or more sequences that each mutually encode multiple genetic elements in the same stretch of sequence (a “co-encoding”). The computational method encodes each of the genetic elements in respective directed acyclic graphs (DAGs) or finite automatons (FAS), then encodes overlapping sequences between the DAGs or FAs in a second DAG or FA. Additional DAGs or FAs may be encoded for overlapping sequences that result from shifting the reading frame of the genetic elements relative to one another and switching the orientation of the elements.

A US patent 2,023,0076603 relates to method and systems for genome sequence compression. The systems and methods for genome sequence compression and decompression are provided. The method for compression encoding of a genome sequence includes partitioning a genome sequence into a plurality of Group of Bases (GoBs) and processing each of the plurality of GoBs independently to encode the genome sequence into a bit stream. Processing each of the plurality of GoBs includes dividing each of the plurality of GoBs into a first part and a second part, the first part including an initial context part and the second part including a learning-based inference part. The processing each of the plurality of GoBs further includes encoding the first part in accordance with a Markov model, encoding the second part in accordance with a learning-based model, and encoding the encoded first part and the encoded second part into the bit stream with an arithmetic encoder. The learning-based model may include Long and Short-Term Memory (LSTM)-based neural networks.

A CN patent 1,060,21985A provides a genome data compression method. The method comprises the steps of performing modeling by using second-generation Illumina sequencing data; extracting key information; and compressing DNA data volume on the premise of not influencing a genome coverage degree by utilizing the key information. According to the method, the data is subjected to data volume compression before the data is analyzed, so that the resource consumption for subsequent analysis is greatly reduced.

By looking at prior art multiple advancements have been seen in similar regards. However, there is always needed advancement in methods and systems to encode and decode genome sequences that provide compact compression and transmission of genome sequences. The present invention pertains to the utilization of information redundancies present between unaligned and aligned genomic data, specifically between a collection of FASTQ files and a SAM/BAM/CRAM file derived from said collection of FASTQ files. Through the application of this invention, a user is able to compress a collection of FASTQ files and the SAM/BAM/CRAM derived from it, thereby achieving a substantially reduced utilization of computer storage and reduced transmission time for sending or receiving said data, in comparison to compressing the FASTQ and SAM/BAM/CRAM files individually.

Furthermore, other desirable features and characteristics will become apparent from the subsequent detailed description and the appended claims, taken in conjunction with this background of the disclosure.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

The primary desirable object of the present invention is to provide a novel and improved method that deals with the compression of unaligned and aligned genomic data.

Another object of the present invention is to provide an advanced mechanism that pertains to the utilization of information redundancies present between unaligned and aligned genomic data, specifically between a collection of FASTQ files and a SAM/BAM/CRAM file derived from said collection of FASTQ files.

Another object of this invention is to provide a new and improved method, wherein a user is able to compress a collection of FASTQ files and the SAM/BAM/CRAM derived from it, thereby achieving a substantially reduced utilization of computer storage and reduced transmission time for sending or receiving said data, in comparison to compressing the FASTQ and SAM/BAM/CRAM files individually.

Thus, it is the objective to provide a new and improved method of genomic compression. Other aspects, advantages and novel features of the present invention will become apparent from the detailed description of the invention when considered in conjunction with the accompanying claims.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

RNA and DNA are essentially chains of 4 possible bases (A,C,G,T in the case of DNA and A,C,G,U in the case of RNA). These chains can be as short as a few hundred bases or shorter as in the case of some RNA molecules or as long as hundreds of millions of bases as in the case of some DNA chromosomes. Scientists and clinicians are often interested in knowing the exact sequence of bases in a particular DNA or RNA molecule. To achieve that, they “sequence” the DNA or RNA molecule in a sequencing machine. However, sequencing machines can only handle relatively short chains of bases (in current technologies, this is between tens of bases to millions of bases depending on the sequencing technology), so the molecule often needs to be first chopped up into a large number of fragments for sequencing. The output of the sequencing process is a computer file which contains “reads”. Each read contains the sequence of bases for one of the fragments. A read looks something like this (the format shown here is FASTQ, that is used by many, but not all, sequencing technologies):

@read-1
NTTGGGG
+
#FFFFFF

The first line consists of a ‘@’ character, the read name and sometimes other metadata. The second line is the actual DNA/RNA sequence of the fragment (the N base means “unknown”). The fourth line are “base qualities”—each character corresponds to the respective base and represents the degree in which the system is confident that the base in the molecule is actually the base listed. The file with the reads typically contains thousands to billions of such reads.

For many species, there exists a “reference genome”, which is a set of published sequences describing a genome derived from one or more individuals of that species. It is common practice to utilize an “aligner” software package, which accepts a collection of unaligned reads and a reference genome, and aligns the unaligned reads against reference genome. The output of an aligner is a file of aligned reads also called alignments. Commonly, aligners output the alignments in one or more of SAM, BAM or CRAM file formats.

An alignment in the SAM format is a tab-separated textual line containing meta data in the header, and a set of alignments. An example of an alignment is:

read-1 97 chr3 58492070 37 51M=58492070 51 NTTGGGG #FFFFFF XT:A:U NM:i:1 SM:i:37

The 1st field is the read name (“read-1”) which may or may not be derived from the read name of the corresponding unaligned read, the 10th field (“NTTGGGG”) is the sequence of the read, and is often either identical to the sequence of the corresponding unaligned read, or a reverse complement of it. The 11th field (“#FFFFFF”) is the base quality scores string-which if often either identical to the base quality scores string of the corresponding unaligned read or a reverse of it. Similar to a FASTQ file, a SAM/BAM/CRAM file often contains hundreds of millions of alignments.

Users of genomic data often need to store both the unaligned reads (often in FASTQ format) and aligned reads (often in SAM/BAM/CRAM format). Data generated by sequencing any particular biological sample, would often include one or more FASTQ files, and also a SAM/BAM/CRAM file generated from these FASTQ file(s) when mapping it against a particular reference genome. Since the SAM/BAM/CRAM file is partially generated from the FASTQ file, it is obvious that there is a substantial overlap of information between the SAM/BAM/CRAM file and the collection of FASTQ files, but how to exploit such overlapping information for the purpose of better compression is non-obvious, due to also some notable differences between the representation of the information in SAM/BAM/CRAM versus FASTQ, including:

1. It is a common practice to combine align data from FASTQ files into a single SAM/BAM/CRAM file—for example, two FASTQ files that represent paired-end reads.

2. The reads in the FASTQ file are often ordered in some sequential order by which they were produced by the sequencing machine or an upstream software package, whereas the alignments in the SAM/BAM/CRAM file are often sorted by the coordinate of the alignments in the reference genome.

3. A sequence in the SAM/BAM/CRAM file may reversed complemented and the base quality score string may be reversed, relative to the FASTQ data.

4. The SAM/BAM/CRAM file might be missing some reads present in the FASTQ, for example, they might have been filtered out.

5. The SAM/BAM/CRAM file might contain multiple alignments per read-in addition to the primary alignment, additional alignments may exist-known as secondary and supplementary alignments.

6. The read names might differ—for example, a read name in the SAM/BAM/CRAM file may be more concise, or may include addition metadata related to the read, compared to the read name of the corresponding unaligned read in the FASTQ file.

7. The base quality scores string might differ due to processing, for example, by “Base Quality Score Recalibration”.

8. The sequence might differ—for example as a result of a software bug or manual editing.

The present invention is directed to provide an improved method that pertains to the utilization of information redundancies present between unaligned and aligned genomic data, specifically between a collection of FASTQ files and a SAM/BAM/CRAM file derived from said collection of FASTQ files. Through the application of this invention, a user is able to compress a collection of FASTQ files and the SAM/BAM/CRAM derived from it, thereby achieving a substantially reduced utilization of computer storage and reduced transmission time for sending or receiving said data, in comparison to compressing the FASTQ and SAM/BAM/CRAM files individually.

In one embodiment of the present invention, there is provided a context-based genomic compression apparatus that incorporates the invention. The genomic compression apparatus proficiently partitions the data contained within the genomic data into various contexts. Furthermore, the apparatus selectively applies a suitable compression methodology customized to each particular context. Within this embodiment, the present invention enhances the compression of the read name data, sequence data, and base quality score data of the FASTQ files by enabling the representation of these data to reference the information present in the SAM/BAM/CRAM file. By employing this referencing mechanism, the present invention optimizes the compression efficiency of the aforementioned data, leading to improved storage and transmission capabilities.

In said embodiment of the present invention, the invention is implemented in four modules, which are described hereinafter.

A first module is employed during the compression process of the SAM/BAM/CRAM file. During this compression process, specific operations are performed for each primary alignment (i.e. Excluding supplementary and secondary alignments). For the read name, sequence, and base quality scores string of each primary alignment, a 32-bit non-negative integer value is computed using the crc32 algorithm as a hash function. In cases where the alignment has the “reverse complement” flag set, the hash function is applied to the reverse complement of the sequence data and the reverse string of the base quality scores string instead of the sequence and the base quality scores string, respectively, as they appear in the SAM/BAM/CRAM file. These three resulting hash values, combined with the sequential number of the alignment within the SAM/BAM/CRAM file, a “next” field, and a “consumed” flag field, form an alignment entry.

The alignment entries are organized within an alignment entry array. Additionally, to enable efficient access to the alignment entries in RAM by the second module, an index for the alignment entries is created in the form of a hash table. For each alignment entry a certain hash table entry is chosen, which is the hash table entry at the location defined by the lower 31 bits of the sequence hash value. Each entry in the hash table consists of the index in the alignment entry array corresponding to a respective alignment entry. The “next” field of the alignment entries enables the formation of one-directional linked lists linking all alignment table entries which correspond to the same hash entry thereby enabling multiple alignment entries to be indexed by the same hash entry. The “consumed” field is reserved for use by the second module.

By storing only the hash values of the read name, sequence, and base quality score string rather than the complete strings, efficient utilization of RAM resources is achieved during the compression process.

The second module is utilized in the process of compressing each read in each file within the FASTQ collection. During this compression, three hash values are computed, specifically for the read name (the characters on the first line of the FASTQ read, starting at the second character and ending at the first space, carriage return or linefeed characters), sequence, and base quality score string. The same hash functions employed in the first module are utilized for this purpose. Subsequently, a hash table entry from the hash table created by the first module is chosen based on the hash value of the sequence, akin to the first module.

Within the linked list present in the selected hash table entry, all alignment table entries referred to are examined to ascertain whether all three hash values in the alignment table entry correspond to the three hash values calculated for the FASTQ read. If none of the alignment table entries pass this test, it implies that the read is not included in the SAM/BAM/CRAM file. As a result, the read is compressed using the default method of the genomic compression apparatus.

If multiple alignment tests succeed, indicating the existence of two or more distinct primary alignments sharing the same hash values, it becomes uncertain which alignment should be referenced. Consequently, in such cases, the default method of the genomic compression apparatus is employed.

In the event that only one alignment table entry passes the test successfully, an additional check is performed on the “consumed” flag. If the flag is set, it signifies that a FASTQ read, which has already been processed, possesses the same three hash values as the current read, but only one SAM/BAM/CRAM alignment exhibits this hash value. In this scenario, it is known that one of these FASTQ reads is not represented in the SAM/BAM/CRAM file; however, it is unclear which one. To handle this exceedingly rare situation, the compression process is terminated, and the user is advised to attempt compression using the default method of the genomic compression apparatus.

Lastly, if the “consumed” flag is not already set, it is set at this stage, and the read is represented as a reference to the sequential number of the alignment in the SAM/BAM/CRAM file, which is retrieved from the alignment entry.

The third module is employed in the process of decompressing the SAM/BAM/CRAM file. Analogous to the first module, once a primary alignment is reconstructed, the read name, sequence, and base quality scores string are stored in the random-access memory (RAM). These values are stored in an array indexed by the sequential number of the alignment within the SAM/BAM/CRAM file.

The fourth and final module comes into play during the decompression of the FASTQ files within the FASTQ collection. When reconstructing a FASTQ read, if the representation of that read refers to a specific sequential number of the alignment in the SAM/BAM/CRAM file, the read name, sequence, and base quality scores string associated with that alignment, which were stored in RAM by the third module, are retrieved and utilized to reconstruct the FASTQ read.

In an alternative embodiment of the present invention, a different set of three hash functions can be employed for hashing the read name, sequence, and base quality scores string. Specifically, in cases where there are multiple primary alignments sharing the same read name, such as in paired-end data, the read name can be concatenated with an indication of the FASTQ file from which a particular alignment originates, for example whether it is “first” or “last” according to the SAM/BAM/CRAM FLAG field, and this combined string can then be hashed. This would usually result in different hash values for otherwise identical read names.

Another embodiment of the present invention allows the second module to consider a FASTQ read as matching a SAM/BAM/CRAM alignment, even if only the read name and sequence match, while the base quality scores string does not match. This is permissible when it is the only matching alignment and the “consumed” flag is not enabled. This feature enables the usage of this invention even if the quality scores have been altered, for instance, through a Base Quality Score Recalibration process. In such cases, the read name and sequence are stored with reference to the corresponding SAM/BAM/CRAM alignment, while the quality scores string is compressed using the default compression method of the genomic compression apparatus, or alternatively the first module stores the base quality scores string, as encountered in the SAM/BAM/CRAM alignment in RAM, and the second module stores a reference to it, including a base-wise delta between the base quality scores as they appear in the FASTQ vs the SAM/BAM/CRAM data.

Another embodiment of the present invention allows the second module to consider a FASTQ read as matching a SAM/BAM/CRAM alignment, even if only the base quality scores and sequence match, while the read name does not match. This is permissible when it is the only matching alignment and the “consumed” flag is not enabled. This feature enables the usage of this invention even if the read name has been altered.

Another embodiment of the present invention allows the second module to consider a FASTQ read as matching a SAM/BAM/CRAM alignment, even if only just the sequence matches, while the read name and base quality scores string do not match. This is permissible when it is the only matching alignment and the “consumed” flag is not enabled. This feature enables the usage of this invention even if both the read name and the base quality scores string have been altered.

Another embodiment of the present invention allows the second module to consider a FASTQ read name as matching a SAM/BAM/CRAM alignment's QNAME field, if the string starting immediately after the first space character in the first line of a FASTQ read, and ending with the at the next carriage return, linefeed or space of the line, matches the QNAME field.

In another embodiment of the present invention, specifically applicable to genomic compressor apparatuses that partition a large genomic file into multiple blocks for separate compression of each block, the sequential number of the alignment within the SAM/BAM/CRAM file is replaced by an index comprising the block number and the alignment number within the block.

Yet another embodiment involves excluding alignments and reads that contain a “monobase sequence,” which refers to a sequence consisting entirely of identical characters (A, C, G, T, or N), from consideration. Such alignments and reads are compressed using the default method of the genomic compression apparatus. This exclusion is implemented due to the increased likelihood of hash contention when dealing with monobase sequences, as FASTQ and SAM/BAM/CRAM files often contain multiple occurrences of such sequences.

In another embodiment, the third module reduces RAM usage during decompression by compressing the read name, sequence, and base quality scores string of each alignment before storing them in RAM. The fourth module then appropriately decompresses these values before utilizing them.

Similarly, another embodiment focuses on reducing RAM consumption during decompression by having the third module store information necessary for retrieving the sequence from the reference file, based on some or all of the RNAME, POS, and/or CIGAR fields of the SAM/BAM/CRAM alignment or an alignment computed by the genomic compression apparatus itself, instead of storing the entire sequence.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: A novel method for compressing a collection of genomic data consisting of unaligned DNA or RNA reads and aligned sequences, by exploiting information redundancies between said unaligned reads and said aligned sequences, to better compress the said collection of genomic data or a subset thereof.
1.1: As per claim 1, the unaligned genomic reads are represented in FASTQ format
1.2: As per claim 1, the aligned sequences are represented in SAM or BAM or CRAM format.
1.3: As per claim 1, the resulting compressed representation of the collection of genomic data, contains sufficient information to allow a decompressor to reconstruct the original data precisely.
1.4: As per claim 1, while parsing SAM/BAM/CRAM data, information regarding the QNAME, SEQ and/or QUAL fields of certain aligned sequences is stored in memory, along with information describing the location of the aligned sequence in the SAM/BAM/CRAM file, and subsequently, while compressing a FASTQ file, for certain FASTQ reads, said information is considered to determine whether any particular read is represented in an aligned sequence in the SAM/BAM/CRAM file, and if so, in certain cases, create a representation of the FASTQ read which includes a reference to said aligned sequence.
1.4.1: As per claim 1.4, the information regarding the QNAME, SEQ and/or QUAL fields is stored in the form of the output of respective hash functions, whose inputs include the respective field, and a mechanism exists to prevent erroneously associating a FASTQ read with an incorrect SAM/BAM/CRAM alignment, due to a chance equivalence of hash values.
1.4.1.1: As per claim 1.4.1, where in the case of paired-end reads, the input of the hash function of the QNAME data also includes the identity of the mate as derived from the SAM/BAM/CRAM “first segment” and/or “last segment” flags.

2. A novel method for precisely reconstructing a collection of genomic data consisting of unaligned DNA or RNA reads and aligned sequences from a compressed representation of said collection, in which any particular unaligned read might be encoded in a representation which includes a reference to an aligned sequence.
2.1: As per claim 2, the resulting unaligned genomic reads are stored in FASTQ format
2.2: As per claim 2, the resulting aligned sequences are stored in SAM or BAM format.
2.3: As per claim 2, when decompressing the aligned sequences, information regarding some or all of the QNAME, SEQ and/or QUAL fields is stored in memory, then, when decompressing the unaligned reads, for a read whose compressed representation includes reference to an aligned sequence, said read's reconstruction process includes retrieving from memory the QNAME, SEQ and/or QUAL information of the referred aligned sequence.
2.3.1: As per claim 2.3, some or all of the QNAME, SEQ and/or QUAL information stored in memory is stored in a compressed representation.
2.3.1.1: As per claim 2.3.1, where the compressed representation of the SEQ information includes a reference to an external set of genomic sequences, so that when reconstructing the unaligned read's sequence data, it is wholly or partially based on data retrieved from said external set of genomic sequences.
